(19) United States
(12) Patent Application Publication
(10) Pub. No.: US 2019/0231650 A1
(43) Pub. Date: Aug. 1, 2019

(54) LIGHT-TRANSMISSIVE ZIRCONIA SINTERED BODY AND PREPARATION METHOD THEREFOR AND USE THEREOF

(71) Applicant: SHANDONG SINOCERA FUNCTIONAL MATERIAL CO., LTD., Dongying (CN)

(72) Inventors: Aimin YANG, Shandong (CN); Xuekui MO, Dongying (CN); Xibin SONG, Shandong (CN)

(73) Assignee: SHANDONG SINOCERA FUNCTIONAL MATERIAL CO., LTD., Dongying (CN)

(21) Appl. No.: 16/329,637

(22) PCT Filed: Aug. 3, 2017
(86) PCT No.: PCT/CN2017/095746

§ 371 Date: Apr. 2, 2019

(30) Foreign Application Priority Data
Aug. 31, 2016 (CN) 201610793418.1

(51) Int. Cl. A61K 6/02; A61C 13/083; A61C 13/00; A61K 6/00; C04B 35/488; C04B 35/626; C04B 35/634; C04B 35/64

(52) U.S. Cl. CPC A61K 6/024; C04B 2235/608; A61C 13/0022; A61K 6/0008; C04B 35/488; C04B 35/6264; C04B 35/63488; C04B 35/63416; C04B 35/62695; C04B 35/6261; C04B 35/64; C04B 2235/3246; C04B 2235/5409; C04B 2235/5436; C04B 2235/9653; C04B 2235/604; C04B 2235/6567; A61C 13/083

(57) ABSTRACT

Provided is a light-transmissive zirconia sintered body, obtained by preparing a green body from a powder for the light-transmissive zirconia sintered body by means of isostatic pressing, and sintering the green body at a high temperature and normal pressure after degreasing and biscuit firing. The light-transmissive zirconia sintered body is prepared by dispersing zirconia powder in water, adding an appropriate amount of a dispersant and a binder, mixing the mixture to form a slurry, and spray-granulating same, wherein the molar percentage of yttrium oxide in the zirconia powder is 3-5%. The light-transmissive zirconia sintered body can be used for preparing a fixed dental prosthesis.

### CROSS-REFERENCE TO RELATED APPLICATION

The present invention claims the priority to Chinese patent application No. 201610793418.1 filed on Aug. 31, 2016 with the CNIPA, the whole disclosed contents thereof is incorporated herein by reference in its entirety.

### TECHNICAL FIELD

The present invention relates to the field of powder engineering, specifically, to a light-transmissive zirconia sintered body and preparation method therefor and use thereof.

### BACKGROUND ART

Zirconia ceramic is a new type of biological powder with good biocompatibility and excellent mechanical properties, which has become the focus of dental ceramic development.

CN104016677A discloses a light-transmissive zirconia sintered body and preparation method therefor. The zirconia for dental use prepared by the method has a relatively low transmittance, poor overall texture and jade-like appearance, is not aesthetically pleasing, and has certain limitations in application.

### SUMMARY OF THE INVENTION

One purpose of the present invention is to provide a novel light-transmissive zirconia sintered body and preparation method therefor.

Another purpose of the present invention is to provide use of the light-transmissive zirconia sintered body in the preparation of a fixed dental prosthesis.

In order to achieve the purpose of the present invention, the present invention first provides a powder for a light-transmissive zirconia sintered body. The powder is obtained by dispersing zirconia powder in water (pure water), adding an appropriate amount of a dispersant and a binder, mixing well to form a slurry, and then spray-granulating. The powder moisture is controlled at a range of 0.1 to 0.5% by weight during granulation, and the average particle diameter of the granulated particles is 30 to 50 μm.

Wherein, the molar percentage of yttrium oxide in the zirconia powder is 3 to 5%; the dispersant comprises at least one selected from acrylic acid, polyacrylic acid, acrylamide, polyurethane and the like; the binder comprises at least one selected from polyvinyl alcohol, polyethylene glycol, acrylic resin, carboxymethyl cellulose and the like.

The zirconia powder used in the present invention has a BET specific surface area of 5 to 15 m²/g, and an average particle diameter of 0.1 to 1.0 μm.

The zirconia powder can be prepared by hydrothermal synthesis, comprising the following steps:

S1, a soluble zirconium salt and a soluble yttrium salt are mixed at a ratio and dissolved in water, and the pH of the slurry is adjusted to 8 to 10 with ammonia water under stirring;

S2, the above slurry is subjected to hydrothermal synthesis at 140 to 200° C. for 10 to 72 h;

S3, the resultant reaction solution is washed with water and dried to obtain a powder;

S4, the powder is subjected to heat treatment at 800 to 1200° C. for 2 to 5 h, and then grinding and dispersing; and

S5, the dispersed particles are granulated to obtain a powder for a light-transmissive zirconia sintered body.

The molar ratio of the soluble zirconium salt to the soluble yttrium salt in step S1 is 95 to 97:3 to 5, preferably 96:4. The soluble zirconium salt includes zirconium oxychloride, zirconium nitrate, zirconium sulfate and the like. The soluble yttrium salt includes yttrium chloride, yttrium nitrate, yttrium sulfate and the like.

The weight parts of the feedstocks for preparing a powder for the light-transmissive zirconia sintered body of the present invention are as follows: 50 to 100 parts of zirconia powder, 0.1 to 0.5 parts of dispersant, 0.1 to 8.0 parts of binder, and 50 to 100 parts of water. Preferably, the weight parts of the feedstocks are as follows: 100 parts of zirconia powder, 0.2 parts of dispersant, 5 parts of binder, and 100 parts of water.

The present invention also provides a light-transmissive zirconia sintered body prepared from the powder. The sintered body has a three-point bending strength of ≥900 MPa, a tetragonal phase accounting for >90% of the sintered body, a light transmittance of ≥46%, and a material shrinkage rate before and after sintering controlled at 18 to 25%.

The light-transmissive zirconia sintered body of the present invention can be prepared according to the following method: a green body is prepared from the powder by isostatic pressing (holding pressure at 150 to 200 MPa for 10 to 50 s), and the green body is subjected to degreasing and biscuit firing at 900 to 1100° C. for 1 to 4 h, and then subjected to high-temperature sintering in the atmosphere under normal pressure at 1400 to 1550° C. for 1 to 4 h.

In the foregoing method, the density of the green body after isostatic pressing is 2.5 to 3.3 g/cm³.

The present invention also provides the use of the light-transmissive zirconia sintered body in the preparation of a fixed dental prosthesis (including denture).

The present invention further provides a fixed dental prosthesis, such as zirconia denture, prepared from the light-transmissive zirconia sintered body.

The zirconia sintered body provided by the present invention has the characteristics of high light transmittance and high strength, and the prepared dentures have good texture, good jade-like appearance, and are closer to the human teeth. The sintering temperature used in the preparation process is low, and the properties and quality of the product are controllable.

### SPECIFIC MODES FOR CARRYING OUT THE EMBODIMENTS

The following Examples are intended to illustrate the present invention, but are not intended to limit the scope of the present invention. Unless otherwise specified, the technical means used in the examples are conventional means well known to those skilled in the art, and the raw materials used are all commercially available products.

The percent sign “%” involved in the present invention means a percentage by mass unless otherwise specified. However, the percentage of a solution, unless otherwise specified, means the number of grams of the solute contained in 100 mL solution.

### Examples 1 to 5: Powder for a Light-Transmissive Zirconia Sintered Body

The powder for a light-transmissive zirconia sintered body of the present invention can be obtained by the following method:

zirconia powder was dispersed in pure water, an appropriate amount of dispersant and binder were added and blended to form a slurry, and then the powder for preparing the light-transmissive zirconia sintered body was obtained by spray granulation. (Table 1)

TABLE 1
Powder for the light-transmissive zirconia sintered body

| | Example 1 | Example 2 | Example 3 | Example 4 | Example 5 |
|---|---|---|---|---|---|
| Molar percentage of yttrium | 3.8 | 3.9 | 4.0 | 4.1 | 4.2 |
| oxide in zirconia powder | | | | | |
| BET specific surface area of | 12.8 | 11.6 | 10.5 | 9.6 | 8.8 |
| zirconia powder, m²/g | | | | | |
| Average particle diameter | 0.198 | 0.192 | 0.201 | 0.235 | 0.223 |
| of zirconia powder, μm | | | | | |
| Dispersant | Acrylic | Polyacrylic | Polyacrylic | Acrylamide | Polyurethane |
| | acid | acid | acid | | |
| Binder | PEG1000 | PVA124 | PVA205 | PEG400 | PVA105 |
| Weight ratio of zirconia | 100:0.4:2:80 | 50:0.1:0.5:100 | 100:0.2:5:100 | 100:0.3:8:80 | 100:0.5:3:50 |
| powder, dispersant, | | | | | |
| binder and water | | | | | |
| Average particle diameter | 35 | 38 | 42 | 39 | 46 |
| of the powder for a light- | | | | | |
| transmissive zirconia | | | | | |
| sintered body, μm | | | | | |

The zirconia powder was prepared by hydrothermal synthesis method comprising the following steps:

S1, the soluble zirconium salt (zirconium nitrate) and the soluble yttrium salt (yttrium nitrate) were mixed at a ratio and dissolved in water, ammonia water was added slowly under stirring, and the pH of the slurry was adjusted to 8 to 10 with ammonia water;

S2, the above slurry was transferred to a reaction kettle, and subjected to hydrothermal synthesis at 140 to 200° C. for 10 to 72 h;

S3, the resultant reaction solution was washed with water and dried to obtain a powder;

S4, the powder was subjected to heat treatment at 800 to 1200° C. for 2 to 5 h, and then grinding and dispersing; and

S5, the dispersed particles were granulated to obtain a powder for a light-transmissive zirconia sintered body.

### Examples 6 to 10: Light-Transmissive Zirconia Sintered Body and Preparation Method Therefor

The powder obtained in Examples 1 to 5 was subjected to isostatic pressing (holding pressure at 150 to 200 MPa for 10 to 50 s) to obtain a green body, and the green body was subjected to degreasing and biscuit firing at 900 to 1100° C. for 1 to 4 h, and then subjected to high-temperature sintering in the atmosphere under normal pressure at 1400 to 1550° C. for 1 to 4 h, to give a light-transmissive zirconia sintered body.

The various indexes of the obtained sintered body products were shown in Table 2.

TABLE 2
Indexes of the light-transmissive zirconia sintered body products

| | Exam- | Exam- | Exam- | Exam- | Exam- |
|---|---|---|---|---|---|
| | ple 6 | ple 7 | ple 8 | ple 9 | ple 10 |
| Density of the | 2.8 | 2.9 | 3.0 | 3.1 | 3.2 |
| green body, g/cm³ | | | | | |
| Three-point flexural | 1080 | 1030 | 1020 | 1000 | 1000 |
| strength, Mpa | | | | | |
| Light transmittance, % | 46.1 | 46.2 | 46.5 | 46.7 | 46.6 |
| Shrinkage of the material | 23.5 | 22.8 | 21.6 | 20.3 | 19.5 |
| after sintering relative | | | | | |
| to before sintering, % | | | | | |
| Percentage of the | 98% | 96% | 99% | 97% | 94% |
| tetragonal phase | | | | | |

The light-transmissive zirconia sintered body provided in the present invention can be used as dental powder for fixed dental prosthesis and the like, such as a denture.

Although the present invention is described in detail with general description and specific embodiments as above, it will be apparent to a person skilled in the art that some modifications and improvements can be made. Therefore, such modifications or improvements made without departing from the spirit of the present invention are intended to be within the scope of the present invention.

### INDUSTRIAL APPLICABILITY

The light-transmissive zirconia sintered body provided in the present invention can be used for preparing a fixed dental prosthesis. The zirconia sintered body provided by the present invention has the characteristics of high light transmittance and high strength, and the prepared dentures have good texture, good jade-like appearance, and are closer to the human teeth.

1.-10. (canceled)

11. A powder for a light-transmissive zirconia sintered body, characterized in that:
the powder is obtained by dispersing zirconia powder in water, adding an appropriate amount of a dispersant and a binder, mixing well to form a slurry, and then spray-granulating;
wherein:
the molar percentage of yttrium oxide in the zirconia powder is 3 to 5%;
the dispersant comprises at least one dispersant selected from the group consisting of acrylic acid, polyacrylic acid, acrylamide, and polyurethane; and
the binder comprises at least one binder selected from the group consisting of polyvinyl alcohol, polyethylene glycol, acrylic resin, and carboxymethyl cellulose.

12. The powder according to claim 11, characterized in that the zirconia powder has a BET specific surface area of 5 to 15 m²/g, and an average particle diameter of 0.1 to 1.0 μm.

13. The powder according to claim 11, characterized in that the zirconia powder is obtained by hydrothermal synthesis, the hydrothermal synthesis comprising the following steps:
S1, a soluble zirconium salt and a soluble yttrium salt are mixed at a molar ratio and dissolved in water, and the pH of the slurry is adjusted to 8 to 10 with ammonia water under stirring;
S2, the above slurry is subjected to hydrothermal synthesis at 140 to 200° C. for 10 to 72 h;
S3, the resultant reaction solution is washed with water and dried to obtain a powder;
S4, the powder is subjected to heat treatment at 800 to 1200° C. for 2 to 5 h, and then grinding and dispersing; and
S5, the dispersed particles are granulated to obtain a powder for a light-transmissive zirconia sintered body;
the molar ratio of the soluble zirconium salt to the soluble yttrium salt in step S1 is 95 to 97:3 to 5.

14. The powder according to claim 13, wherein the molar ratio of the soluble zirconium salt to the soluble yttrium salt is 96:4.

15. The powder according to claim 11, characterized in that weight parts of feedstocks for preparing the powder are as follows: 50 to 100 parts of zirconia powder, 0.1 to 0.5 parts of dispersant, 0.1 to 8.0 parts of binder, and 50 to 100 parts of water.

16. The powder according to claim 11, characterized in that the powder moisture is controlled at a range of 0.1% to 0.5% by weight during granulation, and the average particle diameter of the granulated particles is 30 to 50 μm.

17. A light-transmissive zirconia sintered body prepared from the powder according to claim 11.

18. The light-transmissive zirconia sintered body according to claim 17, characterized in that the sintered body has a three-point bending strength of ≥900 MPa, a tetragonal phase accounting for >90% of the sintered body, a light transmittance of ≥46%, and a material shrinkage rate before and after sintering controlled at 18 to 25%.

19. A method for preparing the light-transmissive zirconia sintered body according to claim 17, characterized in that a green body is prepared from the powder by isostatic pressing, and the green body is subjected to degreasing and biscuit firing at 900 to 1100° C. for 1 to 4 h, and then subjected to high-temperature sintering in the atmosphere under normal pressure at 1400 to 1550° C. for 1 to 4 h.

20. The method according to claim 19, characterized in that the isostatic pressing is performed under the following condition: holding pressure at 150 to 200 MPa for 10 to 50 s; and the density of the green body after isostatic pressing is 2.5 to 3.3 g/cm³.

21. Use of the light-transmissive zirconia sintered body of claim in the preparation of a fixed dental prosthesis.

22. A method of preparing a fixed dental prosthesis, the method comprising:
using the light-transmissive zirconia sintered body of claim 17 to prepare a fixed dental prosthesis.

23. A fixed dental prosthesis prepared using the light-transmissive zirconia sintered body of claim 17.

24. A method of obtaining the powder of claim 11 according to hydrothermal synthesis, the method comprising:
mixing a soluble zirconium salt and a soluble yttrium salt at a molar ratio, the molar ratio of the soluble zirconium salt to the soluble yttrium salt being 95 to 97:3 to 5;
dissolving the mixed soluble zirconium salt and soluble yttrium salt in water to form a slurry;
adjusting a pH of the slurry to 8 to 10 with ammonia water under stirring;
subjecting the slurry to hydrothermal synthesis at 140 to 200° C. for 10 to 72 h;
washing the resultant reaction solution with water drying the resultant reaction solution to obtain a powder;
subjecting the powder to heat treatment at 800 to 1200° C. for 2 to 5 h;
grinding and dispersing the powder; and
granulating dispersed particles to obtain a powder for a light-transmissive zirconia sintered body.

25. A method of preparing a light-transmissive zirconia sintered body, the method comprising:
preparing a light-transmissive zirconia sintered body from the powder according to claim 11.
